(19) United States
(12) Patent Application Publication
(10) Pub. No.: US 2021/0275581 A1
(43) Pub. Date: Sep. 9, 2021

(54) Oral Care Composition Containing Cetylpyridinium Tetrachlorozincate

(71) Applicant: Colgate-Palmolive Company, New York, NY (US)

(72) Inventors: Viktor Dubovoy, Cresskill, NJ (US); Long Pan, Somerset, NJ (US)

(73) Assignee: Colgate-Palmolive Company, New York, NY (US)

(21) Appl. No.: 17/249,489

(22) Filed: Mar. 3, 2021

Related U.S. Application Data
(60) Provisional application No. 62/986,017, filed on Mar. 6, 2020.

(51) Int. Cl. A61K 33/30; A61P 31/04

(52) U.S. Cl. CPC A61K 33/30; A61P 31/04

(57) ABSTRACT

Disclosed herein are oral care compositions comprising cetylpyridinium tetrachlorozincate for use in inhibiting growth of Streptococcus mutans in the oral cavity of a subject. In some embodiments, cetylpyridinium tetrachlorozincate is present in an amount of from 0.0001% to 1%, e.g., from 0.0001% to 0.009%, by weight of the composition.

### CROSS REFERENCE TO RELATED APPLICATIONS

This application claims the benefit and priority of U.S. provisional application 62/986,017, filed on Mar. 6, 2020.

### BACKGROUND

There are typically over 70 different types of bacteria in the mouth and most of them do no harm. However, there are bacteria that can contribute to tooth decay and periodontal disease. Without proper oral hygiene, bacteria in the oral cavity can cause tooth decay and/or periodontal diseases. Bacteria is also one of the major contributors to malodor in the oral cavity. Streptococcus mutans is commonly found in the human oral cavity and is a significant contributor to tooth decay. Streptococcus mutans metabolizes sucrose to lactic acid. The acidic environment created in the mouth by this process causes tooth enamel to be vulnerable to decay.

Antibacterial agents are commonly incorporated into oral care compositions to destroy or retard the growth of bacteria that may cause dental plaque, caries or dental decay, or bad breath. Many antibacterial agents are cationic in order to interact with the negatively-charged microbial cell membranes. Cetylpyridinium chloride (CPC) is a cationic quaternary ammonium compound used in oral care compositions. CPC is known to be effective in preventing dental plaque and reducing gingivitis. Zinc compounds are fairly common ingredients for use in oral care compositions. In these products, zinc compounds are utilized as an antibacterial ingredient to prevent gum inflammation. Commonly used zinc compounds are zinc citrate, zinc lactate, zinc oxide, and zinc nitrate.

While the prior art discloses the use of various antibacterial oral care compositions, there is still a need for additional compositions and methods that provide improved antibacterial efficacy.

### BRIEF SUMMARY

In an aspect, the invention provides a method of inhibiting growth of Streptococcus mutans in the oral cavity of a subject, comprising applying an oral care composition comprising cetylpyridinium tetrachlorozincate to the oral cavity. In some embodiments, cetylpyridinium tetrachlorozincate is present in an amount of from 0.0001 to 1%, e.g., from 0.0001 to 0.009%, by weight of the composition. In some embodiments, the subject is in need of inhibiting growth of Streptococcus mutans in the oral cavity. In certain embodiments, the subject suffers from a periodontal disease such as gingivitis and periodontitis.

In another aspect, the invention provides an oral care composition comprising cetylpyridinium tetrachlorozincate, wherein cetylpyridinium tetrachlorozincate is present in an amount of from 0.0001% to 0.009% by weight of the composition. In some embodiments, cetylpyridinium tetrachlorozincate is present in an amount of from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition. In some embodiments, the composition is a dentifrice, a toothpaste, a gel, a mouthwash, or a mouth rinse.

In another aspect, the invention provides an oral care composition, e.g., any oral care composition disclosed herein, for use in inhibiting bacterial growth, e.g., growth of Streptococcus mutans, in the oral cavity of a subject, comprising cetylpyridinium tetrachlorozincate, wherein cetylpyridinium tetrachlorozincate is present in an amount of from 0.0001 to 1%, e.g., from 0.0001 to 0.009%, by weight of the composition.

In another aspect, the invention provides the use of cetylpyridinium tetrachlorozincate for the making of an oral care composition for inhibiting bacterial growth, e.g., growth of Streptococcus mutans, in the oral cavity of a subject. In some embodiments, cetylpyridinium tetrachlorozincate is present in an amount of from 0.0001 to 1%, e.g., from 0.0001 to 0.009%, by weight of the composition.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The invention relates to an oral care composition comprising cetylpyridinium tetrachlorozincate for inhibiting growth of Streptococcus mutans in the oral cavity of a subject. As used herein, cetylpyridinium tetrachlorozincate ((CP)₂ZnCl₄) means a complex of cetylpyridinium chloride (CPC) with zinc chloride (ZnCl₂), having a structural formula of [(C₂₁H₃₈N)₂][ZnCl₄]. This complex has been described, for example, in WO2019/125829 and WO2019/125792, and each incorporated by reference in its entirety. In this disclosure, the term CPC-ZnCl₂ complex is sometimes used to refer to cetylpyridinium tetrachlorozincate. Cetylpyridinium tetrachlorozincate is not a mere mixture of CPC and ZnCl₂, but involves a covalently or ionically-bound complex.

Cetylpyridinium tetrachlorozincate may be formed by the combination of CPC and ZnCl₂ aqueous solutions and may be a solid precipitate formed by the combination of CPC and ZnCl₂ aqueous solutions. For example, cetylpyridinium tetrachlorozincate powder may be prepared as follows: a 25 weight % CPC solution is prepared by dissolving 2.50 grams of anhydrous CPC in 10.01 grams of deionized water and a 75 weight % ZnCl₂ solution is prepared by dissolving 3.66 grams of anhydrous ZnCl₂ in 4.90 grams of deionized water. 1.0 grams of the 75 weight % ZnCl₂ solution is then added dropwise to 3.76 grams of the 25 weight % CPC solution to obtain a Zn/CPC molar ratio of 2. The 75 weight % ZnCl₂ solution immediately precipitates upon contact with the 25 weight % CPC solution to produce cetylpyridinium tetrachlorozincate complex. Subsequently, the material may be recrystallized from acetone to obtain a pure cetylpyridinium tetrachlorozincate material.

In the present invention, it has been found that cetylpyridinium tetrachlorozincate exhibits superior antibacterial activity than zinc chloride or CPC. In particular, it has been found that the Minimum Inhibitory Concentration (MIC) of cetylpyridinium tetrachlorozincate for Streptococcus mutans is lower than that of zinc chloride or CPC. Streptococcus mutans is a significant contributor to tooth decay. Thus, the use of cetylpyridinium tetrachlorozincate in oral care products would be beneficial.

The invention provides, in an aspect, a method (Method 1.0) of inhibiting growth of Streptococcus mutans in the oral cavity of a subject, comprising applying an oral care composition comprising cetylpyridinium tetrachlorozincate to the oral cavity.

For example, the invention includes:
1.1. Method 1.0, wherein cetylpyridinium tetrachlorozincate is present in an amount of from 0.0001% to 1%, from 0.0001% to 0.009%, from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition
1.2. Method 1.0 or 1.1, wherein the composition comprises a fluoride ion source.
1.3. Method 1.2, wherein the fluoride ion source is selected from sodium fluoride, stannous fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and a combination thereof.
1.4. Method 1.2 or 1.3, wherein the fluoride ion source is present in an amount sufficient to supply 25 ppm to 5,000 ppm of fluoride ions, generally at least 500 ppm, e.g., 500 to 2000 ppm, e.g., 1000 ppm to 1600 ppm, e.g., 1450 ppm.
1.5. Any of Methods 1.2-1.4, wherein the fluoride ion source is sodium fluoride.
1.6. Any of the foregoing methods, wherein the composition comprises a basic amino acid in free or salt form.
1.7. Method 1.6, wherein the basic amino acid comprises one or more of arginine, lysine, citrulline, ornithine, creatine, histidine, diaminobutanoic acid, diaminopropionic acid, salts thereof, or combinations thereof.
1.8. Method 1.6 or 1.7, wherein the basic amino acid has the L-configuration.
1.9. Any of Methods 1.6-1.8, wherein the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.
1.10. Any of Methods 1.6-1.9, wherein the basic amino acid comprises arginine.
1.11. Method 1.10, wherein the basic amino acid comprises L-arginine.
1.12. Method 1.10 or 1.11, wherein the basic amino acid comprises arginine bicarbonate, arginine phosphate, arginine sulfate, arginine hydrochloride or combinations thereof, optionally wherein the basic amino acid is arginine bicarbonate.
1.13. Any of the foregoing methods, the composition comprises an additional zinc ion source other than cetylpyridinium tetrachlorozincate.
1.14. Method 1.13, wherein the additional zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof
1.15. Method 1.13 or 1.14, wherein the additional zinc ion source is present an amount of from 0.01% to 5%, e.g., 0.1% to 4%, or 0.5% to 3%, by weight of the composition.
1.16. Any of Methods 1.13-1.15, wherein the additional zinc ion source is selected from the group consisting of zinc oxide, zinc citrate, and a combination thereof.
1.17. Any of Methods 1.13-1.16, wherein the additional zinc ion source is a combination of zinc oxide and zinc citrate.
1.18. Any of Methods 1.13-1.17, wherein zinc oxide is present in an amount of 0.5% to 2%, e.g., 0.5% to 1.5%, or about 1% by weight of the composition.
1.19. Any of Methods 1.13-1.18, wherein zinc citrate is present in an amount of 0.1% to 1%, 0.25 to 0.75%, or about 0.5% by weight of the composition.
1.20. Any of the preceding methods wherein composition comprises one or more surfactants, e.g., selected from anionic, cationic, zwitterionic, and nonionic surfactants, and mixtures thereof
1.21. Method 1.20, wherein the composition comprises an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS) by weight of the composition.
1.22. Method 1.20 or 1.21, wherein the composition comprises a zwitterionic surfactant, for example a betaine surfactant, for example cocamidopropyl betaine, e.g., in an amount of 0.1%-4.5% by weight, e.g., 0.5-2% cocamidopropyl betaine by weight of the composition.
1.23. Any of the preceding Method, wherein the composition comprises a thickener.
1.24. Method 1.23, wherein the thickener comprises xanthan gum, optionally wherein xanthan gum is present in an amount of from 0.1 to 1%, from 0.2-0.8%, from 0.3% to 0.6%, from 0.3% to 0.5%, or about 0.4% by weight of the composition.
1.25. Method 1.23 or 1.24, wherein the thickener comprises carboxymethyl cellulose, optionally wherein carboxymethyl cellulose is present in an amount of from 0.5% to 2%, from 0.8% to 1.5%, from 1% to 1.3%, from 1% to 1.2% or about 1.1% by weight of the composition.
1.26. Any of Methods 1.23 to 1.25, wherein the thickener comprises xanthan gum in an amount of from 0.1 to 1%, from 0.2-0.8%, from 0.3% to 0.6%, from 0.3% to 0.5%, or about 0.4% by weight of the composition and carboxymethyl cellulose in an amount of from 0.5% to 2%, from 0.8% to 1.5%, from 1% to 1.3%, from 1% to 1.2% or about 1.1% by weight of the composition.
1.27. Any of Methods 123 to 1.26, wherein the thickener comprises xanthan gum present in an amount of from 0.3% to 0.5% by weight of the composition and carboxymethyl cellulose in in an amount of from 1% to 1.2% by weight of the composition.
1.28. Any of Methods 1.23 to 1.27, wherein the thickener further comprises a thickening silica, optionally wherein the thickening silica is present in an amount of from 5 to 10%, from 6% to 8% or about 7%, by weight of the composition, further optionally wherein the thickening silica is present in an amount of from 6% to 8% by weight of the composition.
1.29. Any of the preceding methods, wherein the composition comprises an abrasive.
1.30. Method 1.29, wherein the abrasive is selected from silica abrasives, calcium phosphate abrasives, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, and combinations thereof
1.31. Method 1.29 or 1.30, wherein the abrasive is present in an amount of from 10% to 70%, e.g., from 10% to 30%, e.g., 10% to 20%, 15% to 25%, from 20% to 50%, from 25% to 45%, or from 30% to 40% by weight of the composition.
1.32. Any of Methods 1.29 to 1.31, wherein the abrasive comprises a silica abrasive.
1.33. Composition 1.32, wherein the silica abrasive is present in an amount of from 10% to 30%, e.g., 10% to 20%, 15% to 25%, or about 15%, by weight of the composition.
1.34. Any of the preceding methods, wherein the composition comprises a humectant, optionally wherein the humectant is selected from sorbitol, glycerin and a mixture thereof.
1.35. Method 1.34, wherein the humectant comprises glycerin, optionally wherein glycerin is present in an amount of from 15% to 40%, from 20% to 40%, from 30% to 40%, or about 35% by weight of the composition.
1.36. Method 1.34, wherein the humectant comprises sorbitol, optionally wherein sorbitol is present in an amount of from 15% to 40%, from 20% to 40%, from 30% to 40%, or about 35% by weight of the composition.
1.37. Any of the preceding methods, wherein the composition comprises a stannous ion source, optionally wherein the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof.
1.38. Any of the preceding methods, wherein the composition comprises one or more soluble phosphate salts, e.g. selected from tetrasodium pyrophosphate (TSPP), sodium tripolyphosphate (STPP) and a combination thereof, optionally wherein the one or more soluble phosphate salts are present in an amount of 1-20%, e.g., 1-10%, 1-5%, 5-10%, 2-8%, or 1-3%, e.g., about 2%, by weight of the composition.
1.39. Any of the preceding methods, wherein the composition comprises water, optionally wherein water is present in an amount of about 10% to about 90%, from 10% to 80%, from 20% to 60%, from 20% to 40%, from 10% to 30%, from 20% to 30% or from 25% to 35% by weight of the composition.
1.40. Any of the foregoing methods, wherein the composition comprises an effective amount of one or more antibacterial agents in addition to cetylpyridinium tetrachlorozincate, for example comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising triclosan or cetylpyridinium chloride.
1.41. Any of the preceding methods, wherein the composition comprises a whitening agent, e.g., a selected from the group consisting of peroxides, metal chlorites, perborates, percarbonates, peroxyacids, hypochlorites, and combinations thereof.
1.42. Any of the preceding methods, wherein the composition comprises hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate);
1.43. Any of the preceding methods, wherein the composition comprises an agent that interferes with or prevents bacterial attachment, e.g., solbrol or chitosan.
1.44. Any of the preceding methods, wherein the composition comprises a soluble calcium salt, e.g., selected from calcium sulfate, calcium chloride, calcium nitrate, calcium acetate, calcium lactate, and combinations thereof
1.45. Any of the preceding methods, wherein the composition comprises a physiologically or orally acceptable potassium salt, e.g., potassium nitrate or potassium chloride, in an amount effective to reduce dentinal sensitivity.
1.46. Any of the preceding methods, wherein the composition comprises a breath freshener, fragrance or flavoring.
1.47. Any of the preceding methods, further comprising an oral care ingredient selected from: a film; a colorant; a pH modifying agent; and a sensitivity reducing agent.
1.48. Any of the preceding methods, wherein the composition is a dentifrice, a toothpaste, a gel, a mouthwash, a mouth rinse, a powder, a cream, a strip, a gum, bead, film, or floss.
1.49. Method 1.48, wherein the composition is a toothpaste.
1.50. Method 1.48, wherein the composition is a gel.
1.51. Method 1.48, wherein the composition is a mouthwash.
1.52. Any of the preceding methods, wherein the subject is in need of inhibiting growth of Streptococcus mutans in the oral cavity, optionally wherein a higher level of Streptococcus mutans is present in the oral cavity of the subject, compared to a reference subject (e.g., person having a healthy mouth condition, for example, person who does not suffer from a periodontal disease such as gingivitis and periodontitis).
1.53. Method 1.52, wherein the level of Streptococcus mutans in the oral cavity of the subject is more than 10% higher than the reference subject, e.g., more than 20%, more than 30%, more than 40%, more than 50%, more than 60%, more than 70%, more than 80%, more than 90%, more than 100%, or more than 200%, higher than the reference subject.
1.54. Method 1.52 or 1.53, wherein the subject in need of inhibiting growth of Streptococcus mutans in the oral cavity suffers from a periodontal disease such as gingivitis and periodontitis.

The invention provides, in another aspect, an oral care composition (Composition 2.0) that comprises cetylpyridinium tetrachlorozincate, wherein cetylpyridinium tetrachlorozincate is present in an amount of from 0.0001% to 0.009% by weight of the composition.

For example, the invention includes:
2.1. Composition 2.0, wherein cetylpyridinium tetrachlorozincate is present in an amount of from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition
2.2. Composition 2.0 or 2.1, wherein the composition comprises a fluoride ion source.
2.3. Composition 2.2, wherein the fluoride ion source is selected from sodium fluoride, stannous fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and a combination thereof.
2.4. Composition 2.2 or 2.3, wherein the fluoride ion source is present in an amount sufficient to supply 25 ppm to 5,000 ppm of fluoride ions, generally at least 500 ppm, e.g., 500 to 2000 ppm, e.g., 1000 ppm to 1600 ppm, e.g., 1450 ppm.
2.5. Any of Compositions 2.2-2.4, wherein the fluoride ion source is sodium fluoride.
2.6. Any of the foregoing compositions, wherein the composition comprises a basic amino acid in free or salt form.
2.7. Composition 2.6, wherein the basic amino acid comprises one or more of arginine, lysine, citrulline, ornithine, creatine, histidine, diaminobutanoic acid, diaminopropionic acid, salts thereof, or combinations thereof.
2.8. Composition 2.6 or 2.7, wherein the basic amino acid has the L-configuration.
2.9. Any of Compositions 2.6-2.8, wherein the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.
2.10. Any of Compositions 2.6-2.9, wherein the basic amino acid comprises arginine.
2.11. Composition 2.10, wherein the basic amino acid comprises L-arginine.
2.12. Composition 2.10 or 2.11, wherein the basic amino acid comprises arginine bicarbonate, arginine phosphate, arginine sulfate, arginine hydrochloride or combinations thereof, optionally wherein the basic amino acid is arginine bicarbonate.
2.13. Any of the foregoing compositions, the composition comprises an additional zinc ion source other than cetylpyridinium tetrachlorozincate.
2.14. Composition 2.13, wherein the additional zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof
2.15. Composition 2.13 or 2.14, wherein the additional zinc ion source is present an amount of from 0.01% to 5%, e.g., 0.1% to 4%, or 0.5% to 3%, by weight of the composition.
2.16. Any of Compositions 2.13-2.15, wherein the additional zinc ion source is selected from the group consisting of zinc oxide, zinc citrate, and a combination thereof.
2.17. Any of Compositions 2.13-2.16, wherein the additional zinc ion source is a combination of zinc oxide and zinc citrate.
2.18. Any of Compositions 2.13-2.17, wherein zinc oxide is present in an amount of 0.5% to 2%, e.g., 0.5% to 1.5%, or about 1% by weight of the composition.
2.19. Any of Compositions 2.13-2.18, wherein zinc citrate is present in an amount of 0.1% to 1%, 0.25 to 0.75%, or about 0.5% by weight of the composition.
2.20. Any of the preceding compositions wherein composition comprises one or more surfactants, e.g., selected from anionic, cationic, zwitterionic, and nonionic surfactants, and mixtures thereof
2.21. Composition 2.20, wherein the composition comprises an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS) by weight of the composition.
2.22. Composition 2.20 or 2.21, wherein the composition comprises a zwitterionic surfactant, for example a betaine surfactant, for example cocamidopropyl betaine, e.g., in an amount of 0.1%-4.5% by weight, e.g., 0.5-2% cocamidopropyl betaine by weight of the composition.
2.23. Any of the preceding compositions, wherein the composition comprises a thickener.
2.24. Composition 2.23, wherein the thickener comprises xanthan gum, optionally wherein xanthan gum is present in an amount of from 0.1 to 1%, from 0.2-0.8%, from 0.3% to 0.6%, from 0.3% to 0.5%, or about 0.4% by weight of the composition.
2.25. Composition 2.23 or 2.24, wherein the thickener comprises carboxymethyl cellulose, optionally wherein carboxymethyl cellulose is present in an amount of from 0.5% to 2%, from 0.8% to 1.5%, from 1% to 1.3%, from 1% to 1.2% or about 1.1% by weight of the composition.
2.26. Any of Compositions 2.23 to 2.25, wherein the thickener comprises xanthan gum in an amount of from 0.1 to 1%, from 0.2-0.8%, from 0.3% to 0.6%, from 0.3% to 0.5%, or about 0.4% by weight of the composition and carboxymethyl cellulose in an amount of from 0.5% to 2%, from 0.8% to 1.5%, from 1% to 1.3%, from 1% to 1.2% or about 1.1% by weight of the composition.
2.27. Any of Compositions 2.23 to 2.26, wherein the thickener comprises xanthan gum present in an amount of from 0.3% to 0.5% by weight of the composition and carboxymethyl cellulose in in an amount of from 1% to 1.2% by weight of the composition.
2.28. Any of Compositions 2.23 to 2.27, wherein the thickener further comprises a thickening silica, optionally wherein the thickening silica is present in an amount of from 5 to 10%, from 6% to 8% or about 7%, by weight of the composition, further optionally wherein the thickening silica is present in an amount of from 6% to 8% by weight of the composition.
2.29. Any of the preceding compositions, wherein the composition comprises an abrasive.
2.30. Composition 2.29, wherein the abrasive is selected from silica abrasives, calcium phosphate abrasives, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, and combinations thereof
2.31. Composition 2.29 or 2.30, wherein the abrasive is present in an amount of from 10% to 70%, e.g., from 10% to 30%, e.g., 10% to 20%, 15% to 25%, from 20% to 50%, from 25% to 45%, or from 30% to 40% by weight of the composition.
2.32. Any of Compositions 2.29 to 2.31, wherein the abrasive comprises a silica abrasive.
2.33. Composition 2.32, wherein the silica abrasive is present in an amount of from 10% to 30%, e.g., 10% to 20%, 15% to 25%, or about 15%, by weight of the composition.
2.34. Any of the preceding compositions, wherein the composition comprises a humectant, optionally wherein the humectant is selected from sorbitol, glycerin and a mixture thereof.
2.35. Composition 2.34, wherein the humectant comprises glycerin, optionally wherein glycerin is present in an amount of from 15% to 40%, from 20% to 40%, from 30% to 40%, or about 35% by weight of the composition.
2.36. Composition 2.34, wherein the humectant comprises sorbitol, optionally wherein sorbitol is present in an amount of from 15% to 40%, from 20% to 40%, from 30% to 40%, or about 35% by weight of the composition.
2.37. Any of the preceding compositions, wherein the composition comprises a stannous ion source, optionally wherein the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof.
2.38. Any of the preceding compositions, wherein the composition comprises one or more soluble phosphate salts, e.g. selected from tetrasodium pyrophosphate (TSPP), sodium tripolyphosphate (STPP) and a combination thereof, optionally wherein the one or more soluble phosphate salts are present in an amount of 1-20%, e.g., 1-10%, 1-5%, 5-10%, 2-8%, or 1-3%, e.g., about 2%, by weight of the composition.
2.39. Any of the preceding compositions, wherein the composition comprises water, optionally wherein water is present in an amount of about 10% to about 90%, from 10% to 80%, from 20% to 60%, from 20% to 40%, from 10% to 30%, from 20% to 30% or from 25% to 35% by weight of the composition.
2.40. Any of the foregoing compositions, wherein the composition comprises an effective amount of one or more antibacterial agents in addition to cetylpyridinium tetrachlorozincate, for example comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising triclosan or cetylpyridinium chloride.
2.41. Any of the preceding compositions, wherein the composition comprises a whitening agent, e.g., a selected from the group consisting of peroxides, metal chlorites, perborates, percarbonates, peroxyacids, hypochlorites, and combinations thereof.
2.42. Any of the preceding compositions, wherein the composition comprises hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate);
2.43. Any of the preceding compositions, wherein the composition comprises an agent that interferes with or prevents bacterial attachment, e.g., solbrol or chitosan.
2.44. Any of the preceding compositions, wherein the composition comprises a soluble calcium salt, e.g., selected from calcium sulfate, calcium chloride, calcium nitrate, calcium acetate, calcium lactate, and combinations thereof
2.45. Any of the preceding compositions, wherein the composition comprises a physiologically or orally acceptable potassium salt, e.g., potassium nitrate or potassium chloride, in an amount effective to reduce dentinal sensitivity.
2.46. Any of the preceding compositions, wherein the composition comprises a breath freshener, fragrance or flavoring.
2.47. Any of the preceding compositions further comprising an oral care ingredient selected from: a film; a colorant; a pH modifying agent; and a sensitivity reducing agent.
2.48. Any of the preceding compositions, wherein the composition is a dentifrice, a toothpaste, a gel, a mouthwash, a mouth rinse, a powder, a cream, a strip, a gum, bead, film, or floss.
2.49. Composition 2.48, wherein the composition is a toothpaste.
2.50. Composition 2.48, wherein the composition is a gel.
2.51. Composition 2.48, wherein the composition is a mouthwash.
2.52. Any of the forgoing compositions for use to inhibit bacteria growth in the oral cavity of a subject.
2.53. Any of the forgoing compositions for use to inhibit the growth of Streptococcus mutans in the oral cavity of a subject.
2.54. Composition 2.53, wherein the subject is in need of inhibiting growth of Streptococcus mutans in the oral cavity, optionally wherein a higher level of Streptococcus mutans is present in the oral cavity of the subject, compared to a reference subject (e.g., person having a healthy mouth condition, for example, person who does not suffer from a periodontal disease such as gingivitis and periodontitis).
2.55. Composition 2.54, wherein the level of Streptococcus mutans in the oral cavity of the subject is more than 10% higher than the reference subject, e.g., more than 20%, more than 30%, more than 40%, more than 50%, more than 60%, more than 70%, more than 80%, more than 90%, more than 100%, or more than 200%, higher than the reference subject.
2.56. Composition 2.54 or 2.55, wherein the subject in need of inhibiting growth of Streptococcus mutans in the oral cavity suffers from a periodontal disease such as gingivitis and periodontitis.

The oral care composition of the invention can be in the form of any oral care formulations, including dentifrice, toothpaste, gel, mouthwash, mouth rinse, powder, cream, strip, gum, bead, film, floss or any other known in the art. In some embodiments, the oral care composition is a toothpaste or gel. In some embodiments, the oral care composition is a mouthwash or mouth rinse.

The oral care composition of the invention may be a single phase oral care composition. For example, all the components of the oral care composition may be maintained together with one another in a single phase and/or vessel. For example, all the components of the oral care composition may be maintained in a single phase, such as a single homogenous phase. In another embodiment, the oral care composition may be a multi-phase oral care composition.

The oral care composition of the invention may contain an orally acceptable carrier. As used herein, an “orally acceptable carrier” refers to a material or combination of materials that are safe for use in the compositions of the invention, commensurate with a reasonable benefit/risk ratio. Such materials include but are not limited to, for example, water, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof. Such materials are well known in the art and are readily chosen by one skilled in the art based on the physical and aesthetic properties desired for the compositions being prepared. In some embodiment, the orally acceptable carrier may include an orally acceptable solvent. Illustrative solvents may include, but are not limited to, one or more of ethanol, phenoxyethanol, isopropanol, water, cyclohexane, methyl glycol acetate, benzyl alcohol, or the like, or any mixture or combination thereof. In a particular embodiment, the orally acceptable solvent includes benzyl alcohol.

Water may be present in the oral compositions of the invention. Water employed in the preparation of commercial oral compositions should be deionized and free of organic impurities. Water commonly makes up the balance of the compositions and includes about 10% to about 90%, about 10% to about 80%, about 20% to about 60%, about 20% to 40%, about 10% to about 30%, about 20% to 30%, or about 25% to 35% by weight of the oral compositions. This amount of water includes the free water which is added plus that amount which is introduced with other materials such as with sorbitol or any components of the invention.

The oral care composition of the invention comprises cetylpyridinium tetrachlorozincate in an amount of from 0.0001% to 1%, e.g., from 0.0001% to 0.009% by weight of the composition. In some embodiments, cetylpyridinium tetrachlorozincate may be present in an amount of from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition.

The oral care composition of the invention may comprise a basic amino acid in free or salt form. The basic amino acids which can be used in the compositions include not only naturally, occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrulline, ornithine, creatine, histidine, diaminobutanoic acid, diaminopropionic acid, salts thereof or combinations thereof. In a particular embodiment, the basic amino acids are selected from arginine, lysine, citrulline, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate. In some embodiments, the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.

The oral care composition of the invention may comprise an additional zinc ion source other than cetylpyridinium tetrachlorozincate. The additional zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc propionate, zinc salicylate, zinc silicate, zinc stearate, zinc tartrate, zinc undecylenate, and mixtures thereof. In some embodiments, the additional zinc ion source is present in an amount of from 0.01% to 5%, e.g., 0.1% to 4%, or 1% to 3%, by weight of the composition.

In some embodiments, the additional zinc ion source is selected from zinc oxide, zinc citrate, and a combination thereof. Zinc oxide may be present in an amount of 0.5% to 2%, e.g., 0.5% to 1.5%, or about 1% by weight of the composition. Zinc citrate may be present in an amount of 0.1% to 1%, 0.25% to 0.75%, about 0.5% by weight of the composition by weight of the composition. In some embodiments, the composition comprises zinc oxide and zinc citrate. The composition may comprise zinc oxide in an amount of 0.5% to 2%, e.g., 0.5% to 1.5%, about 1% or about 1.2% by weight of the composition and zinc citrate in an amount of 0.1% to 1%, 0.25% to 0.75%, about 0.5% by weight of the composition. In certain embodiments, the composition comprises zinc oxide in an amount of about 1% by weight of the composition and zinc citrate in an amount of about 0.5% by weight of the composition.

The oral care composition of the invention may include a stannous ion source. The stannous ion source can be a soluble or an insoluble compound of stannous with inorganic or organic counter ions. Examples include the fluoride, chloride, chlorofluoride, acetate, hexafluorozirconate, sulfate, tartrate, gluconate, citrate, malate, glycinate, pyrophosphate, metaphosphate, oxalate, phosphate, carbonate salts and oxides of stannous. In some embodiments, the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glyoxide, and mixtures thereof.

The oral care composition of the invention may include fluoride, such as one or more fluoride ion sources (e.g., soluble fluoride salts). A wide variety of fluoride ion-yielding materials may be employed as sources of soluble fluoride. Illustrative fluoride ion sources include, but are not limited to, sodium fluoride, stannous fluoride, potassium fluoride, sodium monofluorophosphate, fluorosilicate salts, such as sodium fluorosilicate and ammonium fluorosilicate, amine fluoride, ammonium fluoride, and combinations thereof. In some embodiment, the fluoride ion source includes sodium fluoride. The amount of the fluoride ion source present in the oral care composition may be greater than 0 weight % and less than 0.8 wt. %, less than 0.7 wt. %, less than 0.6 wt. %, less than 0.5 wt. %, or less than 0.4 wt. %. The fluoride ion sources may be present in an amount sufficient to supply 25 ppm to 5,000 ppm of fluoride ions, generally at least 500 ppm, e.g, 500 to 2000 ppm, e.g., 1000 ppm to 1600 ppm, e.g., 1450 ppm.

The oral care composition of the invention may include thickeners. Suitable thickeners may be any orally acceptable thickener or thickening agent configured to control the viscosity of the oral care composition. Illustrative thickeners may be or include, but are not limited to, colloidal silica, fumed silica, a cross-linked polyvinylpyrrolidone (PVP) polymer, cross-linked polyvinylpyrrolidone (PVP), or the like, or mixtures or combinations thereof. In some embodiments, the thickening system includes a cross-linked polyvinylpyrrolidone (PVP) polymer. The thickening system may also include POLYPLASDONE® XL 10F, which is commercially available from Ashland Inc. of Covington, Ky. Illustrative thickeners may also be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, or the like, or mixtures or combinations thereof. Thickeners particularly suitable of use in the oral care composition of the invention include natural and synthetic gums and colloids. Optionally, the composition comprises at least one gum selected from carrageenan and xanthan gum.

In some embodiments, the composition comprises xanthan gum. Xanthan gum may be present in an amount of from 0.1 to 1%, from 0.2-0.8%, from 0.3% to 0.6%, from 0.3% to 0.5%, or about 0.4% by weight of the composition. In some embodiments, the composition comprises carboxymethyl cellulose. Carboxymethyl cellulose may be present in an amount of from 0.5% to 2%, from 0.8% to 1.5%, from 1% to 1.3%, from 1% to 1.2% or about 1.1% by weight of the composition. In some embodiments, the composition comprises xanthan gum in an amount of from 0.1 to 1%, from 0.2-0.8%, from 0.3% to 0.6%, from 0.3% to 0.5%, or about 0.4% by weight of the composition and carboxymethyl cellulose in an amount of from 0.5% to 2%, from 0.8% to 1.5%, from 1% to 1.3%, from 1% to 1.2% or about 1.1% by weight of the composition. In certain embodiments, the composition comprises xanthan in an amount of from 0.3% to 0.5% by weight of the composition and carboxymethyl cellulose in in an amount of from 1% to 1.2% by weight of the composition. In some embodiments, the composition comprises a thickening silica, optionally wherein the thickening silica is present in an amount of from 5 to 10%, from 6% to 8% or about 7%, by weight of the composition. In some embodiments, the composition comprises xanthan gum present in an amount of from 0.1 to 1%, from 0.2-0.8%, from 0.3% to 0.6%, from 0.3% to 0.5%, or about 0.4% by weight of the composition, carboxymethyl cellulose in in an amount of from 0.5% to 2%, from 0.8% to 1.5%, from 1% to 1.3%, from 1% to 1.2% or about 1.1% by weight of the composition, and a thickening silica in an amount of from 5 to 10%, from 6% to 8% or about 7%, by weight of the composition. In certain embodiments, the composition comprises xanthan gum present in an amount of from 0.3% to 0.5% by weight of the composition, carboxymethyl cellulose in in an amount of from 1% to 1.2% by weight of the composition, and a thickening silica in an amount of from 6% to 8% by weight of the composition.

In some embodiments, the oral care compositions may include one or more abrasives or an abrasive system including one or more abrasives. As used herein, the term “abrasive” may also refer to materials commonly referred to as “polishing agents”. Any orally acceptable abrasive may be used, but preferably, type, fineness (particle size), and amount of the abrasive may be selected such that the tooth enamel is not excessively abraded in normal use of the oral care composition. The one or more abrasives may have a particle size or D50 of less than or equal to about 10 μm, less than or equal to about 8 μm, less than or equal to about 5 μm, or less than or equal to about 3 μm. The one or more abrasives may have a particle size or D50 of greater than or equal to about 0.01 μm, greater than or equal to about 0.05 μm, greater than or equal to about 0.1 μm, greater than or equal to about 0.5 μm, or greater than or equal to about 1 μm. Illustrative abrasives may include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate (e.g., precipitated calcium carbonate and/or natural calcium carbonate), magnesium carbonate, hydrated alumina, silica, zirconium silicate, aluminum silicate including calcined aluminum silicate, polymethyl methacrylate, or the like, or mixtures and combinations thereof. In some embodiments, the oral care composition comprises a silica abrasive. In some embodiments, the silica abrasive is present in an amount of from 10% to 30%, e.g., 10% to 20%, 15% to 25%, or about 15%, by weight of the composition. In some embodiments, the oral care composition comprises a calcium-free silica abrasive. In some embodiments, the composition is substantially free of calcium, e.g., comprises less than 2%, less than 1%, less than 0.5%, or less than 0.1% calcium by weight of the composition.

In some embodiments, the oral care composition of the invention comprises a calcium-containing abrasive (e.g., calcium carbonate). In some embodiments, the calcium-containing abrasive is selected from calcium carbonate, calcium phosphate (e.g., dicalcium phosphate dihydrate), calcium sulfate, and combinations thereof. In some embodiments, the oral care composition comprises calcium carbonate as an abrasive. In one embodiment, the oral care composition comprises precipitated calcium carbonate or natural calcium carbonate. Precipitated calcium carbonate may be preferred over natural calcium carbonate.

The amount or concentration of the one or more abrasives present in the oral care composition may vary widely. In some embodiments, the amount of the abrasives present in the oral care composition may be from about 15 weight % to about 70 weight %, e.g., from about 20 weight % to about 50 weight %, from about 25 weight % to about 45 weight %, from about 30 weight % to about 40 weight %, from about 10% to about 20 weight %, or about 15 weight %, based on a total weight of the oral care composition.

The oral care composition of the present invention may include at least one surfactant or solubilizer. Suitable surfactants include neutral surfactants (such as polyoxyethylene hydrogenated castor oil or fatty acids of sugars), anionic surfactants (such as sodium lauryl sulfate), cationic surfactants (such as the ammonium cation surfactants) or zwitterionic surfactants. These surfactants or solubilizers may be present in amounts of typically from 0.01% to 5%, from 0.01% to 2%; or from 1% to 2%; or about 1.5%, by weight of the composition. In some embodiments, the composition may comprise an anionic surfactant. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium lauryl ether sulfate, ammonium lauryl sulfate, ammonium lauryl ether sulfate, sodium cocoyl monoglyceride sulfonate, sodium lauryl sarcosinate, sodium lauryl isethionate, sodium laureth carboxylase, and sodium dodecyl benzenesulfonate. In some embodiments, the anionic surfactant, e.g., sodium lauryl sulfate (SLS), is present in an amount of from about 0.3% to about 4.5% by weight, e.g. 1-2% by weight of the composition. In some embodiments, the composition may comprise a betaine zwitterionic surfactant. The betaine zwitterionic surfactant may be a C₈-C₁₆ aminopropyl betaine, e.g., cocamidopropyl betaine. In some embodiments, the betaine zwitterionic surfactant, e.g., cocamidopropyl betaine, is present in an amount of from 1% to 1.5%, from 1.1% to 1.4%, from 1.2% to 1.3%, or about 1.25% by weight of the composition. In some embodiments, the composition may comprise a non-ionic block copolymer. The non-ionic block copolymer may be a poly(propylene oxide)/poly(ethylene oxide) copolymer. In some embodiments, the copolymer has a polyoxypropylene molecular mass of from 3000 to 5000 g/mol and a polyoxyethylene content of from 60 to 80 mol %. In some embodiments, the non-ionic block copolymer is a poloxamer. In some embodiments, the non-ionic block copolymer is selected from: Poloxamer 338, Poloxamer 407, Poloxamer, 237, Poloxamer, 217, Poloxamer 124, Poloxamer 184, Poloxamer 185, and a combination of two or more thereof.

In some embodiments, the oral care composition of the invention may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In a preferred embodiment, the orally acceptable vehicle may be or include, but is not limited to, glycerin or sorbitol. In some embodiments, the humectant is selected from glycerin, sorbitol and a combination thereof. In some embodiments, the humectant may be present in an amount of from 20% to 60%, for example from 15% to 40%, from 15% to 35%, from 20% to 40%, from 30% to 50%, from 30% to 40%, or from 40% to 45%, by weight of the composition. In some embodiments, the composition comprises glycerin, optionally wherein glycerin is present in an amount of from 15% to 40%, from 20% to 40%, from 30% to 40%, or about 35% by weight of the composition. In some embodiments, the composition comprises sorbitol, optionally wherein sorbitol is present in an amount of from 15% to 40%, from 20% to 40%, from 30% to 40%, or about 35% by weight of the composition.

The oral care composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof.

The oral care composition of the present invention may include a sweetener such as, for example, saccharin, for example sodium saccharin, acesulfam, neotame, cyclamate or sucralose; natural high-intensity sweeteners such as thaumatin, stevioside or glycyrrhizin; or such as sorbitol, xylitol, maltitol or mannitol. One or more of such sweeteners may be present in an amount of from 0.005% to 5% by weight, for example 0.01% to 1%, for example 0.01% to 0.5%, by weight of the composition.

The oral care composition of the present invention may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is typically incorporated in the oral composition at a concentration of 0.01 to 3% by weight.

The oral care composition of the invention may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or the like, or combinations thereof.

The oral care composition of the invention may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof. Sodium phosphate may include monosodium phosphate (NaH₂PO₄), disodium phosphate (Na₂HPO₄), trisodium phosphate (Na₃PO₄), and mixtures thereof. In a typical embodiment, the buffering agent may be anhydrous sodium phosphate dibasic or disodium phosphate and/or sodium phosphate monobasic. In another embodiment, the buffering agent includes anhydrous sodium phosphate dibasic or disodium phosphate, and phosphoric acid (e.g., syrupy phosphoric acid; 85%-Food Grade).

The oral care composition of the invention may include anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent includes tetrasodium pyrophosphate (TSPP), sodium tripolyphosphate (STPP), or a combination thereof.

The oral care composition of the invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

The oral care composition of the invention may include one or more pigments, such as whitening pigments. In some embodiments, the whitening pigments include particles ranging in size from about 0.1 μm to about 10 μm with a refractive index greater than about 1.2. Suitable whitening agents include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof. The whitening pigment, such as titanium dioxide particles, may be present in an amount that is sufficient to whiten the teeth.

All ingredients for use in the compositions described herein should be orally acceptable. As used herein, “orally acceptable” may refer any ingredient that is present in a composition as described in an amount and form which does not render the composition unsafe for use in the oral cavity.

The invention provides a method of inhibiting bacterial growth in the oral cavity of a subject, comprising applying an oral care composition as disclosed herein to the oral cavity. In some embodiments, the method inhibits growth of Streptococcus mutans in the oral cavity of a subject. In some embodiments, the subject is in need of inhibiting growth of Streptococcus mutans in the oral cavity, optionally wherein a higher level of Streptococcus mutans is present in the oral cavity of the subject, compared to a reference subject (e.g., person having a healthy mouth condition, for example, person who does not suffer from a periodontal disease such as gingivitis and periodontitis). In some embodiments, the level of Streptococcus mutans in the oral cavity of the subject is more than 10% higher than the reference subject, e.g., more than 20%, more than 30%, more than 40%, more than 50%, more than 60%, more than 70%, more than 80%, more than 90%, more than 100%, or more than 200%, higher than the reference subject. In certain embodiments, the subject in need of inhibiting growth of Streptococcus mutans in the oral cavity suffers from a periodontal disease such as gingivitis and periodontitis.

The method of the invention may include contacting the oral care composition with water. The method may also include contacting the surface of the teeth with the oral care composition. Contacting the surface of the teeth with the oral care composition may include disposing the oral care composition (e.g., toothpaste) on a toothbrush and brushing the teeth with the toothbrush. The oral care composition may be applied and/or contacted with the surfaces of the teeth at predetermined intervals. For example, a daily basis, at least once a day, twice a day, or more, for multiple days, or alternatively every other day. In another example, the oral care composition may be applied and/or contacted with the surfaces of the teeth at least once a day, at least once every two days, at least once every three days, at least once every five days, at least once a week, at least once every two weeks, or at least once a month. The oral care composition thereof may be utilized for up to 2 weeks, up to 3 weeks, up to 4 weeks, up to 6 weeks, up to 8 weeks, or greater.

The invention further provides an oral care composition, e.g., any oral care composition disclosed herein, e.g., any of Compositions 2 et seq., for use in inhibiting bacterial growth, e.g., growth of Streptococcus mutans, in the oral cavity of a subject, comprising cetylpyridinium tetrachlorozincate, wherein cetylpyridinium tetrachlorozincate is present from 0.0001 to 1%, e.g., from 0.0001 to 0.009%, by weight of the composition. In some embodiments, cetylpyridinium tetrachlorozincate may be present in an amount of from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition.

The invention further provides the use of cetylpyridinium tetrachlorozincate for the making of an oral care composition for use in inhibiting bacterial growth, e.g., growth of Streptococcus mutans, in the oral cavity of a subject, wherein cetylpyridinium tetrachlorozincate is present in an amount of from 0.0001% to 1%, e.g., from 0.0001% to 0.009% by weight of the composition. In some embodiments, cetylpyridinium tetrachlorozincate may be present in an amount of from 0.0001% to 0.008%, from 0.0001% to 0.007%, from 0.0001% to 0.006%, from 0.0001% to 0.005%, from 0.0001% to 0.004%, from 0.0001% to 0.003%, from 0.0001% to 0.002%, from 0.0001% to 0.001%, from 0.0002% to 0.009%, from 0.0002% to 0.008%, from 0.0002% to 0.007%, from 0.0002% to 0.006%, from 0.0002% to 0.005%, from 0.0002% to 0.004%, from 0.0002% to 0.003%, from 0.0002% to 0.002%, from 0.0002% to 0.001%, from 0.0003% to 0.009%, from 0.0003% to 0.008%, from 0.0003% to 0.007%, from 0.0003% to 0.006%, from 0.0003% to 0.005%, from 0.0003% to 0.004%, from 0.0003% to 0.003%, from 0.0003% to 0.002%, from 0.0003% to 0.001%, from 0.0004% to 0.009%, from 0.0004% to 0.008%, from 0.0004% to 0.007%, from 0.0004% to 0.006%, from 0.0004% to 0.005%, from 0.0004% to 0.004%, from 0.0004% to 0.003%, from 0.0004% to 0.002%, from 0.0004% to 0.001%, from 0.0005% to 0.009%, from 0.0005% to 0.008%, from 0.0005% to 0.007%, from 0.0005% to 0.006%, from 0.0005% to 0.005%, from 0.0005% to 0.004%, from 0.0005% to 0.003%, from 0.0005% to 0.002%, from 0.0005% to 0.001%, from 0.0006% to 0.009%, from 0.0006% to 0.008%, from 0.0006% to 0.007%, from 0.0006% to 0.006%, from 0.0006% to 0.005%, from 0.0006% to 0.004%, from 0.0006% to 0.003%, from 0.0006% to 0.002%, or from 0.0006% to 0.001%, by weight of the composition.

### Examples

### Preparation of Cetylpyridinium Tetrachlorozincate

Cetylpyridinium tetrachlorozincate powder was prepared as follows: a 25 weight % CPC solution was prepared by dissolving 2.50 grams of anhydrous CPC in 10.01 grams of deionized water and a 75 weight % ZnCl₂ solution was prepared by dissolving 3.66 grams of anhydrous ZnCl₂ in 4.90 grams of deionized water. 1.0 grams of the 75 weight % ZnCl₂ solution was then added dropwise to 3.76 grams of the 25 weight % CPC solution to obtain a Zn/CPC molar ratio of 2. The 75 weight % ZnCl₂ solution immediately precipitated upon contact with the 25 weight % CPC solution to produce the CPC-ZnCl₂ complex. Subsequently, the cetylpyridinium tetrachlorozincate material was recrystallized from acetone to obtain a pure cetylpyridinium tetrachlorozincate material.

### Antimicrobial Assays

Salmonella enterica Serovar typhimurium LT2, Staphylococcus aureus USA300 LAC, and Streptococcus mutans Clark (ATCC) were used for the experiment. Salmonella enterica serovar typhimurium is a primary enteric pathogen affecting humans. S. aureus LAC is a community-associated methicillin-resistant CA-MRSA strain and a representative strain of the USA300 clone, which is a leading cause of skin and soft tissue infections in North America. S. mutans is also gram-positive and a leading cause of dental caries. S. enterica and S. aureus were cultured in Muller Hinton media (Sigma-Aldrich) and S. mutans was cultured in Reinforced Clostridial Media (Oxoid). Stock solutions of ZnCl₂ (500 mg/mL); Cetylpyridinium chloride (CPC; 1 mg/mL) and Cetylpyridinium tetrachlorozincate ((CP)₂ZnCl₄; 1 mg/mL) were prepared by dissolving the compounds in distilled and deionized water and filter sterilization prior to use.

Single bacterial colonies were inoculated into 2 mL of medium in 10 mL capacity culture tubes. Inoculated cultures of S. aureus and S. enterica were grown aerobically at 37° C. with shaking at 200 rpm for 24 hours. S. mutans was cultured statically for 48 hours. End-point Minimum inhibitory Concentration (MIC) were determined for ZnCl₂, CPC and (CP)₂ZnCl₄ using Broth Microdilution Method standard protocol from Clinical and Laboratory Standards Institute (M07-A8 Volume 29 No. 2; 2009), Overnight cultures in triplicate were standardized to 0.5 McFarland standards (OD₆₀₀=0.1). MICs were determined in cultures grown in 96 well microtiter plates. 100 μL of the standardized culture was sub-cultured into wells containing 100 μL of medium containing antimicrobial compound. Control wells containing 200 μL of media only or media compound were used to standardize the data. The microtiter plates were aerobically incubated statically at 37° C. for 20 hours. Culture optical densities (A₆₀₀) were determined using Biotek® EPOCH 2 microplate reader.

The ability of Zn and (CP)₂ZnCl₄ to inhibit the growth of bacterial pathogens was examined. The minimal inhibitory concentrations of Zn and (CP)₂ZnCl₄ were determined in liquid culture after static growth. All three bacteria displayed typical dose-responses to the compounds utilized. The MICs of Zn and (CP)₂ZnCl₄ are shown in Table 1.

TABLE 1
The minimal inhibitory concentrations of ZnCl₂ and (CP)₂ZnCl₄

| | S. aureus | S. enterica | S. mutans |
|---|---|---|---|
| | (μg/mL) | (μg/mL) | (μg/mL) |
| ZnCl₂ | 200 | 300 | 65 |
| (CP)₂ZnCl₄ | 6 | 60 | 6 |

The MICs of ZnCl₂ for S. aureus, S. enterica, and S. mutans were approximately 200, 300, and 65 μg/mL, respectively, whereas the MICs of (CP)₂ZnCl₄ for S. aureus, S. enterica, and S. mutans were 6, 60, and 6 μg/mL, respectively. The ability of CPC to inhibit the growth of bacterial pathogens was further examined. The MICs of CPC were similar to those of (CP)₂ZnCl₄ for S. aureus and S. enterica. However, (CP)₂ZnCl₄ had a slight improvement in antibacterial activity against S. mutans, compared to CPC. These results show a superior antibacterial efficacy of (CP)₂ZnCl₄ against S. mutans.

While the disclosure has been described with respect to specific examples including presently preferred modes of carrying out the disclosure, those skilled in the art will appreciate that there are numerous variations and permutations of the above described systems and techniques. It is to be understood that other embodiments may be utilized and structural and functional modifications may be made without departing from the scope of the present disclosure. Thus, the scope of the disclosure should be construed broadly as set forth in the appended claims.

1. A method of inhibiting growth of Streptococcus mutans in the oral cavity of a subject, comprising applying an oral care composition comprising cetylpyridinium tetrachlorozincate to the oral cavity.

2. The method of claim 1, wherein cetylpyridinium tetrachlorozincate is present in an amount of from 0.0001% to 1% by weight of the composition.

3. The method of claim 2, wherein cetylpyridinium tetrachlorozincate is present in an amount of from 0.0001% to 0.009% by weight of the composition.

4. The method of claim 1, wherein the composition comprises a fluoride ion source.

5. The method of claim 1, wherein the composition comprises an additional zinc ion source other than cetylpyridinium tetrachlorozincate.

6. The method of claim 1, wherein the composition comprises a stannous ion source.

7. The method of claim 1, wherein the composition comprises a basic amino acid, in free or salt from.

8. The method of claim 1, wherein the composition comprises a surfactant.

9. The method of claim 1, wherein the subject is in need of inhibiting growth of Streptococcus mutans in the oral cavity.

10. An oral care composition comprising cetylpyridinium tetrachlorozincate, wherein cetylpyridinium tetrachlorozincate is present in an amount of from 0.0001% to 0.009% by weight of the composition.

11. The composition of claim 10, wherein cetylpyridinium tetrachlorozincate is present in an amount of from 0.0006% to 0.006% by weight of the composition.

12. The composition of claim 10, wherein the composition comprises a fluoride ion source.

13. The composition of claim 10, wherein the composition comprises an additional zinc ion source other than cetylpyridinium tetrachlorozincate.

14. The composition of claim 10, wherein the composition comprises a stannous ion source.

15. The composition of claim 10, wherein the composition comprises a basic amino acid, in free or salt from.

16. The composition of claim 10, wherein the composition comprises a surfactant.

17. The composition of claim 10 for use in inhibiting growth of Streptococcus mutans in the oral cavity of a subject.

18. (canceled)
